(19) United States
(12) Patent Application Publication
(10) Pub. No.: US 2026/0306709 A1
(43) Pub. Date: Oct. 8, 2026

(54) ORDERED COLLAGEN SCAFFOLD MATERIAL, PREPARATION METHOD THEREFOR, AND USE THEREOF

(71) Applicant: DB WUDEREGEN BIOMEDICAL TECHNOLOGIES (JIANG SU) Co., Ltd, Zhenjiang (CN)

(72) Inventors: Qun DONG, Zhenjlang (CN); Hengfei REN, Zhenjiang (CN); Wei ZHANG, Zhenjiang (CN)

(21) Appl. No.: 19/098,100

(22) Filed: Apr. 2, 2025

(30) Foreign Application Priority Data
Feb. 5, 2024 (CN) 202410161754.9

(51) Int. Cl. A61L 27/24; A61L 27/36; A61L 27/38

(52) U.S. Cl. CPC A61L 27/24; A61L 27/3687; A61L 27/3691; A61L 27/3878; A61L 2430/32; A61L 2430/40

(57) ABSTRACT

The present disclosure provides an ordered collagen scaffold material, a preparation method therefor and use thereof. The preparation method includes: subjecting a mammalian fascia tissue to a combination treatment of electrical pulse stimulation and ultrasound stimulation; soaking with an alkaline solution; soaking in an organic solution for a preliminary degreasing treatment, and then performing a secondary degreasing treatment with supercritical carbon dioxide; performing a cross-linking treatment with a chemical cross-linking agent; fixing and molding; and lyophilizing to obtain the ordered collagen scaffold material. The product prepared by the preparation method has an ordered parallel arrangement of collagen fiber structure, which retains the nerve cell adhesion ability and nerve fiber extension ability of collagen, and removes the residue of organic substances and reagents in the product, so as to induce the effective regeneration of the nervous tissue after spinal cord injury.

### CROSS-REFERENCE TO RELATED APPLICATION(S)

This application claims priority to Chinese Patent Application No. 202410161754.9, filed Feb. 5, 2024, the disclosure of which is incorporated herein by reference in its entirety.

### TECHNICAL FIELD

The present application relates to the technical field of medical biomaterials, and more specifically, to an ordered collagen scaffold material, a preparation method therefor and use thereof.

### BACKGROUND

Spinal cord is the main carrier of motion signal and sensory signal transmission between brain and body. After spinal cord injury, ischemia and hypoxia, immunoreaction, free radical release and other pathological reactions lead to nerve cell death, axon and myelolysis, and the nerve conduction function is destroyed, the regeneration inhibition microenvironment is formed at the injury site, so that the sensory and motor nerve conduction function is difficult to be restored, which ultimately causes paralysis of the patient, and imposes a huge burden on the family and the society. After the spinal cord injury, the ordered structure of the tissue is destroyed and the injury site is replaced by a cavity or scar that creates a physical barrier for nerve regeneration, which is one of the challenges in repairing spinal cord injuries.

Biomaterials are used to bridge the two injury ends to guide the growth of nerve fibers, which is essential for repairing the spinal cord injury. Collagen is the most abundant extracellular matrix in the body and can be degraded to amino acids by a variety of enzymes in the body. Moreover, collagen, as an extracellular matrix, can transmit exogenous signals to the cells via transmembrane proteins such as integrins and glycoproteins, and regulate or induce adhesion, migration, and growth of cells, providing a good medium for nerve regeneration. Collagenous materials are used to prepare a fiber scaffold with an ordered arrangement is of great significance for repairing spinal cord injury.

CN106256380A discloses a use of a functional biomaterial in the preparation of a material that promotes the repair of the total spinal cord transection injury in animals. The functional biomaterial is a biomaterial obtained by modifying a nerve regeneration collagen scaffold with collagen-binding brain-derived neurotrophic factor; the collagen-binding brain-derived neurotrophic factor is a protein with an amino acid sequence of SEQ ID No. 1; the neural regeneration collagen scaffold is prepared as follows: (1) treating the fascia with tributyl phosphate to obtain a TnBP-treated material; (2) treating the TnBP-treated material with NaCl to obtain an NaCl-treated material; and (3) treating the NaCl-treated material with trypsin to obtain a nerve regeneration collagen scaffold.

In most existing technical methods, conventional decellularization reagents such as SDS (sodium dodecyl sulfate), sodium deoxycholate, tributyl phosphate, and Triton X-100 are required to remove cells, while enzymes are employed to eliminate residual proteins. However, these conventional decellularization reagents struggle to rapidly penetrate deep into tissues, resulting in not only prolonged decellularization time and suboptimal efficacy but also a high risk of collagen denaturation during tissue processing, which compromises their neural regeneration outcomes. Although the use of enzymes such as trypsin, papain, elastase, and collagenase facilitates tissue loosening, their usage inevitably causes varying degrees of damage to collagen structures. This leads to reduced adhesion and extension efficiency of nerve cells on the material and may also impair in vivo stability.

Therefore, it is of great significance to develop a product that can effectively remove cells, residual proteins, and lipids from tissues, reduces the solvent residue risk, and improves the adhesion efficiency and efficiency of repairing nerve cells.

### SUMMARY

The present application provides an ordered collagen scaffold material, a preparation method therefor, and use thereof. The product prepared by this preparation method has an ordered parallel arrangement of collagen fiber structure, which retains the nerve cell adhesion ability and nerve fiber extension ability of collagen, and removes the residue of organic substances and reagents in the product, so as to induce the effective regeneration of the nervous tissue after spinal cord injury.

In a first aspect, the present application provides a preparation method for an ordered collagen scaffold material, and the preparation method includes:
(1) subjecting a mammalian fascia tissue to a combination treatment of electrical pulse stimulation and ultrasound stimulation;
(2) soaking a product after treatment in an alkaline solution;
(3) soaking a product after alkali solution treatment in an organic solution for a preliminary degreasing treatment, and performing a secondary degreasing treatment with supercritical carbon dioxide;
(4) cross-linking a product after degreasing treatment with a chemical cross-linking agent;
(5) fixing and molding a product after cross-linking treatment; and
(6) lyophilizing a product after fixing and molding to obtain the ordered collagen scaffold material.

The present application innovatively develops a collagen scaffold material for spinal cord injury repair and a preparation method therefor. This method employs a dual physical field system combining ultrasound and electrical pulses for tissue pretreatment. The synergistic enhancement between these two stimulation modalities significantly improves decellularization efficiency compared to individual ultrasound or electrical pulse treatments, consequently dramatically enhancing spinal cord repair efficacy. Moreover, the combined approach does not require the addition of conventional decellularization reagents, greatly reducing the risk of solvent residue and collagen denaturation during processing, resulting in not only shorter decellularization time but also better effectiveness. At the same time, the combined approach also eliminates the need for enzymatic treatment, thereby avoiding the issue of collagen structure being easily damaged by the use of enzymes, which would reduce the adhesion and extension efficiency of nerve cells, resulting in not only shorter decellularization time but also better effectiveness.

The method employs a combination of organic solvents and supercritical carbon dioxide for efficient degreasing, which not only more effectively degreases from fascial tissue, but also further reduces the risk of solvent residue. Additionally, the process is relatively mild and does not affect the neural repair efficiency of the collagen scaffold.

On the basis of chemical cross-linking treatment, the method also introduces fixing-molding and lyophilizing treatments, which can further enhance the in vivo stability of the material while achieving structural orderliness, improving the collagen content of the product and the adhesion and growth effects of nerve cells.

In summary, the preparation method of the ordered collagen scaffold material involved in the present application, through the effective coordination of the above steps, greatly improves the efficiency of material preparation, enhances the collagen content and the adhesion and growth effects of nerve cells, and improves the repair effect of spinal cord injury.

Preferably, in step (1), the combination treatment is performed with electrical pulse stimulation and ultrasound stimulation for 1-5 times alternately or sequentially, such as 1 time, 2 times, 3 times, 4 times, or 5 times.

The “for 1-5 times alternately or sequentially” means that the electrical pulse stimulation and the ultrasound stimulation are alternately performed for 1-5 times separately, or the electrical pulse stimulation is first performed for 1-5 times and then the ultrasound stimulation is performed for 1-5 times, or the ultrasound stimulation is first performed for 1-5 times and then the electrical pulse stimulation is performed for 1-5 times.

The electrical pulse stimulation is to treat a fascia tissue with an electrode, and a stimulation voltage is set to 10-100 V (such as 10 V, 20 V, 30 V, 40 V, 50 V, 60 V, 70 V, 80 V, 90 V, or 100 V, etc.), a pulse length is set to 10-250 ms (such as 20 ms, 40 ms, 50 ms, 70 ms, 80 ms, 100 ms, 120 ms, 150 ms, 170 ms, 180 ms, 200 ms, or 250 ms, etc.), and a pulse interval is set to 1-7 s (such as 1 s, 2 s, 3 s, 4 s, 5 s, 6 s, or 7 s, etc.), and 10-100 pulses (such as 10, 20, 30, 40, 50, 60, 70, 80, 90, 100, etc.) are stimulated each time.

The ultrasound stimulation is performed with a power of 50-200 W (such as 50 W, 70 W, 80 W, 100 W, 120 W, 140 W, 150 W, 160 W, 170 W, 180 W, or 200 W, etc.) for a period of 1-10 min (such as 1 min, 2 min, 3 min, 4 min, 5 min, 6 min, 7 min, 8 min, 9 min, or 10 min, etc.).

Preferably, in step (2), the alkali solution is an aqueous NaOH solution of 0.5-3 M (such as 0.5 M, 0.7 M, 0.8 M, 1.0 M, 1.3 M, 1.5 M, 2.0 M, 2.5 M, or 3.0 M, etc.) of, and the soaking is performed for a period of 0.5-3 h (such as 0.5 h, 0.7 h, 0.8 h, 0.9 h, 1.0 h, 1.2 h, 1.4 h, 1.5 h, 2 h, or 3 h, etc.), and the soaking is performed at a temperature of 2-10° C. (such as 2° C., 3° C., 4° C., 5° C., 6° C., 7° C., 8° C., or 10° C., etc.).

A product after soaking is further washed.

Preferably, in step (3), the organic solution includes a combination of ethanol and ethyl acetate, and a volume ratio of ethanol to ethyl acetate is 1:1-1:4, such as 1:1, 1:2, 1:3, or 1:4, etc.

In step (3), the soaking is performed for a period of 15-30 h (such as 15 h, 17 h, 18 h, 19 h, 20 h, 21 h, 22 h, 24 h, 25 h, or 30 h, etc.), and the soaking is performed at a temperature of 20-30° C. (such as 20° C., 22° C., 24° C., 26° C., 28° C., or 30° C., etc.); and a product after soaking is further washed.

In the preparation method of the ordered collagen scaffold material involved in the present application, the organic solvent selected for degreasing is more preferably a combination of ethanol and ethyl acetate. Compared to other degreasing solvents, such as acetone, ether, or other combinations, the combination of ethanol and ethyl acetate exhibits superior degreasing efficacy, the fat content in the product is lower, and the effect of repairing spinal cord injury is better.

Preferably, in the secondary degreasing treatment with supercritical carbon dioxide of step (3), the supercritical carbon dioxide has a pressure of 5-40 MPa (such as 5 MPa, 10 MPa, 15 MPa, 20 MPa, 25 MPa, 30 MPa, 35 MPa, or 40 MPa, etc.) at a temperature of 25-55° C. (such as 25° C., 30° C., 35° C., 40° C., 45° C., 50° C., 55° C., etc.); and carbon dioxide has a flow rate of 0.5-12 L/min (such as 1 L/min, 3 L/min, 4 L/min, 6 L/min, 8 L/min, 10 L/min, or 12 L/min, etc.), and the secondary degreasing treatment is performed for a period of 20-40 min (such as 20 min, 25 min, 30 min, 35 min, or 40 min, etc.).

Preferably, in step (4), the chemical cross-linking agent includes N-hydroxysuccinimide and 1-(3-dimethylaminopropyl)-3-ethyl carbodiimide; and the cross-linking treatment is performed at a temperature of 30-40° C. (such as 30° C., 31° C., 32° C., 33° C., 34° C., 35° C., 37° C., or 39° C., etc.) for a period of 0.5-1.5 h (such as 0.5 h, 0.7 h, 0.8 h, 0.9 h, 1.0 h, 1.2 h, 1.4 h, or 1.5 h, etc.).

A product after cross-linking is further washed.

Preferably, in step (5), the fixing and molding is performed in a way that the product is alternately soaking in ethanol solution and water for a total of 3-8 times (such as 3 times, 4 times, 5 times, 6 times, 7 times, or 8 times), and each soaking is performed for a period of 3-8 min (such as 3 min, 4 min, 5 min, 6 min, 7 min, or 8 min, etc.); wherein an ethanol concentration of the ethanol solution is 75-100% (such as 75%, 80%, 85%, 90%, 95%, or 100%, etc.).

Preferably, in step (6), the lyophilizing is performed firstly by pre-freezing, and a procedure of the lyophilizing is as follows: a first stage: −35 to −25° C. for 1-5 h; a second stage: −25 to −15° ° C. for 0.5-1.5 h; a third stage: −25 to −15° C. for 5-15 h; a fourth stage: −15 to −5° C. for 0.5-1.5 h; a fifth stage: −15 to −5° C. for 10-20 h; a sixth stage: −5 to −5° C. for 1-5 h; a seventh stage: 5 to 15° C. for 1-5 h; an eighth stage: 10 to 20° C. for 1-5 h; a ninth stage: 20 to 30° C. for 1-5 h; and vacuuming is performed in the second stage.

The specific point value in the “−35 to −25° C.” can be selected as −35° C., −32° C., −30° C., −28° C., −27° C., −26° C., or −25° C., etc.; the specific point value in the “1-5 h” can be selected as 1 h, 2 h, 3 h, 4 h, or 5 h, etc.; the specific point value in the “−25 to −15° C.” can be selected as −25° C., −22° C., −20° C., −18° C., −17° C., −16° C., or −15° C., etc.; the specific point value in the “0.5-1.5 h” can be selected as 0.5 h, 0.7 h, 0.8 h, 1 h, 1.2 h, or 1.5 h, etc.; the specific point value in the “5-15 h” can be selected as 5 h, 8 h, 10 h, 12 h, 14 h, 15 h, etc.; the specific point value in the “−15 to −5° C.” can be selected as −15° C., −12° C., −10° C., −8° C., −7° C., −6° C., or −5° C., etc.; the specific point value in the “10-20 h” can be selected as 10 h, 12 h, 14 h, 16 h, 18 h, or 20 h, etc.; the specific point value in the “−5 to 5° C.” can be selected as −5° C., −2° C., −0° C., 2° C., 3° C., 4° C., or 5° C., etc.; the specific point value in the “5 to 15° C.” can be selected as 5° C., 7° C., 8° C., 9° C., 10° C., 12° C., or 15° C., etc.; the specific point value in the “10 to 20° C.” can be selected as 10° C., 12° C., 14° C., 15° C., 16° C., 18° C., or 20° C., etc.; and the specific point value in the “20 to 30° C.” can be selected as 20° C., 22° C., 24° C., 25° C., 26° C., 28° C., or 30° C., etc., Other specific point values in the above value range can be selected and will not be repeated here.

In the preparation method for the ordered collagen scaffold material involved in the present application, the procedure of lyophilizing treatment is also specially designed with a total of nine stages, each stage meets the specific temperature range and time range, so that the structure of the final product can be more ordered, which enhances the in vivo stability of the material and the effect of repairing the spinal cord.

In a second aspect, the present application provides an ordered collagen scaffold material prepared by the preparation method according to the first aspect.

In a third aspect, the present application provides use of the ordered collagen scaffold material according to the second aspect in products for repairing spinal cord injury.

In a fourth aspect, the present application provides a product for repairing spinal cord injury, the product for repairing spinal cord injury includes the ordered collagen scaffold material according to the second aspect and Cetuximab loaded on the ordered collagen scaffold material;

wherein the product for repairing spinal cord injury is prepared by a preparation method including the following steps:

soaking the ordered collagen scaffold material in Cetuximab solution and incubating at 0-30° C. (such as 1° C., 2° C., 3° C., 4° C., 5° C., 6° C., 7° C., 8° C., 9° C., 10° C., 15° C., 20° C., 25° C., or 30° C., etc.) for 20-60 min (such as 20 min, 30 min, 40 min, 50 min, or 60 min, etc.). Other specific point values within the above ranges can be selected and will not be repeated here.

Spinal cord injury triggers the activation and proliferation of glial cells, leading to the formation of glial scars. Studies have shown that reactive astrocytes appearing after spinal cord injury extensively express chondroitin sulfate proteoglycans (CSPGs), a class of molecules that inhibit neural regeneration. Chondroitin sulfate proteoglycans not only spatially hinder axonal regeneration in the form of scars but also activate the protein kinase C (PKC) signaling pathway within neurons, subsequently activating Rho to suppress neural regeneration. Additionally, the activation of the epidermal growth factor receptor (EGFR) triggers the Rho-Rac signaling pathway, further inhibiting axonal regeneration. Currently, the EGFR antibody Cetuximab is clinically used as a therapeutic drug. Research results have confirmed that in the spinal cord injury environment, Cetuximab effectively reduces the deposition of glial scars and their inhibitory molecules, chondroitin sulfate proteoglycans, in the injury area, thereby promoting neural regeneration. Therefore, by utilizing ordered collagen to guide the orderly growth of nerve fibers and combining it with Cetuximab to further reduce scar formation, the two approaches synergize to better reconstruct the injury microenvironment and enhance neural regeneration. The functional neural regeneration collagen scaffold formed by this combination provides a novel therapeutic approach for the clinical treatment of spinal cord injury repair.

Compared with the prior art, the present application has the following beneficial effects.

The ordered collagen scaffold material prepared by the method of the present invention features an orderly parallel-aligned collagen fiber structure, retains collagen's inherent capability for nerve cell adhesion and nerve fiber extension, and effectively eliminates residual organic substances and reagents in the product, enabling it to induce effective regeneration of neural tissue following spinal cord injury. In the method, the dual physical field pretreatment combining ultrasound and electrical pulses achieves synergistic enhancement, significantly improving decellularization efficiency compared to individual ultrasound or electrical pulse treatments; the combination of organic solvent and supercritical carbon dioxide efficiently degreases from fascial tissue, not only enhancing lipid clearance but also reducing solvent residue risks, while maintaining mild processing conditions that preserve neural repair efficacy of the collagen scaffold; and on the basis of chemical cross-linking treatment, the method also introduces fixing-molding and lyophilizing treatments, which can further enhance the in vivo stability of the material while achieving structural orderliness, improving the collagen content of the product and the adhesion and growth effects of nerve cells.

### BRIEF DESCRIPTION OF DRAWINGS

FIG. 1 is a scanning electron micrograph of the product prepared in Example 1.

FIG. 2 is an immunofluorescent staining of neuron cells by Tuj1 antibody.

FIG. 3 is a graph showing the results of glial scar formation in the no-material transplantation group and the Example 1 material transplantation group.

FIG. 4 is a graph showing the results of nerve regeneration in the no-material transplantation group and the Example 1 material transplantation group.

FIG. 5 is a graph showing the results of glial scar formation situation in the transplantation repair group of the Application Example material.

FIG. 6 is a graph showing the results of nerve regeneration in the transplantation repair group of the Application Example material.

### DETAILED DESCRIPTION

The technical solution of the present application is further described below by specific embodiments. It should be clear to those skilled in the art that the examples are merely an aid to understanding the present application and should not be considered as a specific limitation of the present application.

### Example 1

This example provides an ordered collagen scaffold material, and the preparation method of the ordered collagen scaffold material includes the following steps.
(1) A fresh bovine fascial tissue was taken, and the muscle and adipose tissues were physically removed, and then soaked in deionized water at 4° C. for 2 h, with the water being changed every half hour.
(2) After soaking the tissue in water at 4° C. for 15 min, the tissue was subjected to electrical pulse stimulation using electrodes. The stimulation voltage was set at 90 V, with a pulse length of 100 ms and a pulse interval of 2 s, for a total of 50 pulses.
(3) The tissue obtained from step (2) was then soaked in water at 16° C. and subjected to ultrasonic stimulation at a power of 100 W for 5 min.
(4) Steps (2) and (3) were repeated three times.
(5) The tissue obtained from step (4) was soaked in a 2 M NaOH aqueous solution for 1 h, after which the alkaline solution was removed and the tissue was washed with deionized water 10 times, for 5 min each time.
(6) The tissue obtained from step (5) was then soaked in a mixed solution of ethanol and ethyl acetate (with a volume ratio of 1:2) at 16° C. for 10 h, followed by washing with deionized water 10 times, for 5 min each time.
(7) The tissue obtained from step (6) was treated with supercritical carbon dioxide at a pressure of 30 MPa, a temperature of 30° C., and a carbon dioxide flow rate of 6 L/min for 30 min.
(8) The tissue obtained from step (7) was treated with a solution of 1 mg/mL EDC (1-(3-Dimethylaminopropyl)-3-ethylcarbodiimide hydrochloride) and 0.5 mg/mL NHS (N-hydroxysuccinimide) for 1 h, collected, and washed with deionized water 3 times, for 5 min each time.
(9) The tissue obtained from step (8) was soaked in 100% ethanol for 5 min, then the liquid was changed to deionized water for 5 min, and this process was repeated 5 times, and then soaked in deionized water for 30 min.
(10) The tissue obtained from step (9) underwent lyophilizing, first pre-frozen at −70° C. for 4 h, then placed in a lyophilizer. In the second stage, a vacuum was applied, and the lyophilizing process was carried out as follows.

| Stage | Temperature | Time |
|---|---|---|
| 1 | −30 °C | 3 h |
| 2 | −20 °C | 1 h |
| 3 | −20 °C | 10 h |
| 4 | −10 °C | 1 h |
| 5 | −10 °C | 15 h |
| 6 | 0 °C | 2 h |
| 7 | 10 °C | 2 h |
| 8 | 15 °C | 2 h |
| 9 | 25 °C | 2 h |

### Example 2

This example provides an ordered collagen scaffold material, and the preparation method of the ordered collagen scaffold material includes the following steps.
(1) A fresh bovine fascial tissue was taken, and the muscle and adipose tissues were physically removed, and then soaked in deionized water at 4° C. for 2 h, with the water being changed every half hour.
(2) After soaking the tissue in water at 4° C. for 15 min, the tissue was subjected to electrical pulse stimulation using electrodes. The stimulation voltage was set at 90 V, with a pulse length of 200 ms and a pulse interval of 1 s, for a total of 100 pulses.
(3) The tissue obtained from step (2) was then soaked in water at 16° C. and subjected to ultrasonic stimulation at a power of 200 W for 3 min.
(4) Steps (2) and (3) were repeated three times.
(5) The tissue obtained from step (4) was soaked in a 1 M NaOH aqueous solution for 1.5 h, after which the alkaline solution was removed and the tissue was washed with deionized water 10 times, for 5 min each time.
(6) The tissue obtained from step (5) was then soaked in a mixed solution of ethanol and ethyl acetate (with a volume ratio of 1:3) at 16° C. for 15 h, followed by washing with deionized water 10 times, for 5 min each time.
(7) The tissue obtained from step (6) was treated with supercritical carbon dioxide at a pressure of 25 MPa, a temperature of 35° C., and a carbon dioxide flow rate of 3 L/min for 30 min.
(8) The tissue obtained from step (7) was treated with a solution of 1 mg/mL EDC and 0.5 mg/mL NHS at 16° C. for 1 h, collected, and washed with deionized water 3 times, for 5 min each time.
(9) The tissue obtained from step (8) was soaked in 90% ethanol for 7 min, then the liquid was changed to deionized water for 5 min, and this process was repeated 5 times, and then soaked in deionized water for 30 min.
(10) The tissue obtained from step (9) underwent lyophilizing, first pre-frozen at −70° C. for 4 h, then placed in a lyophilizer. In the second stage, a vacuum was applied, and the lyophilizing process was carried out as follows.

| Stage | Temperature | Time |
|---|---|---|
| 1 | −30 °C | 4 h |
| 2 | −20 °C | 1 h |
| 3 | −20 °C | 16 h |
| 4 | −10 °C | 1 h |
| 5 | −10 °C | 20 h |
| 6 | 0 °C | 4 h |
| 7 | 10 °C | 4 h |
| 8 | 15 °C | 4 h |
| 9 | 25 °C | 4 h |

### Example 3

This example provides an ordered collagen scaffold material, and the preparation method of the ordered collagen scaffold material includes the following steps.
(1) A fresh bovine fascial tissue was taken, and the muscle and adipose tissues were physically removed, and then soaked in deionized water at 4° C. for 2 h, with the water being changed every half hour.
(2) After soaking the tissue in water at 4° C. for 15 min, the tissue was subjected to electrical pulse stimulation using electrodes. The stimulation voltage was set at 20 V, with a pulse length of 20 ms and a pulse interval of 5 s, for a total of 10 pulses.
(3) The tissue obtained from step (2) was then soaked in water at 16° C. and subjected to ultrasonic stimulation at a power of 50 W for 7 min.
(4) Steps (2) and (3) were repeated five times.
(5) The tissue obtained from step (4) was soaked in a 2.5 M NaOH aqueous solution for 0.5 h, after which the alkaline solution was removed and the tissue was washed with deionized water 10 times, for 5 min each time.
(6) The tissue obtained from step (5) was then soaked in a mixed solution of ethanol and ethyl acetate (with a volume ratio of 1:1) at 16° C. for 10 h, followed by washing with deionized water 10 times, for 5 min each time.
(7) The tissue obtained from step (6) was treated with supercritical carbon dioxide at a pressure of 40 MPa, a temperature of 40° C., and a carbon dioxide flow rate of 4 L/min for 20 min.
(8) The tissue obtained from step (7) was treated with a solution of 1 mg/mL EDC and 0.5 mg/mL NHS at 16° C. for 1 h, collected, and washed with deionized water 3 times, for 5 min each time.
(9) The tissue obtained from step (8) was soaked in 75% ethanol for 8 min, then the liquid was changed to deionized water for 5 min, and this process was repeated 7 times, and then soaked in deionized water for 30 min.
(10) The tissue obtained from step (9) underwent lyophilizing, first pre-frozen at −70° C. for 4 h, then placed in a lyophilizer. In the second stage, a vacuum was applied, and the lyophilizing process was carried out as follows.

| Stage | Temperature | Time |
|---|---|---|
| 1 | −30 °C | 1 h |
| 2 | −20 °C | 1 h |
| 3 | −20 °C | 8 h |
| 4 | −10 °C | 1 h |
| 5 | −10 °C | 10 h |
| 6 | 0 °C | 1 h |
| 7 | 10 °C | 1 h |
| 8 | 15 °C | 1 h |
| 9 | 25 °C | 1 h |

### Example 4

This example provides an ordered collagen scaffold material, and the preparation method differs from Example 1 only in that an equal volume of ethyl acetate was replaced with acetone in step (6), and other conditions remained unchanged.

### Example 5

This example provides an ordered collagen scaffold material, and the preparation method differs from Example 1 only in that an equal volume of ethanol was replaced with ethyl acetate in step (6), and other conditions remained unchanged.

### Example 6

This example provides an ordered collagen scaffold material, and the preparation method differs from Example 1 only in that the process of lyophilizing was as follows.

| Stage | Temperature | Time |
|---|---|---|
| 1 | −20 °C | 30 h |
| 2 | 25 °C | 8 h |

### Comparative Example 1

This comparative example provides an ordered collagen scaffold material, the preparation method differs from Example 1 only in that the soaking in step (6) was performed for 10 h and 30 min, the treatment in step (7) was not performed, and other conditions remained unchanged.

### Comparative Example 2

This comparative example provides an ordered collagen scaffold material, the preparation method differs from Example 1 only in that the treatment with supercritical carbon dioxide in step (7) was performed for 1 h, the treatment in step (6) was not performed, and other conditions remained unchanged.

### Comparative Example 3

This comparative example provides an ordered collagen scaffold material, the preparation method differs from Example 1 only in that the treatment in step (9) was not performed, and other conditions remain unchanged.

### Comparative Example 4

This comparative example provides an ordered collagen scaffold material, and the preparation method is as follows (a combination treatment of electrical pulse stimulation and ultrasound stimulation was replaced by the treatment with an aqueous solution of tributyl phosphate).
(1) A fresh bovine fascial tissue was taken, and the muscle and adipose tissues were physically removed, and then soaked in deionized water at 4° C. for 2 h, with the water being changed every half hour.
(2) The tissue was treated in a 1% tributyl phosphate aqueous solution for 48 h, and washed with deionized water 5 times, for 10 min each time.
(3) The tissue obtained from step (2) was soaked in a 2 M NaOH aqueous solution for 1 h, after which the alkaline solution was removed and the tissue was washed with deionized water 10 times, for 5 min each time.
(4) The tissue obtained from step (3) was then soaked in a mixed solution of ethanol and ethyl acetate (with a volume ratio of 1:2) at 16° C. for 10 h, followed by washing with deionized water 10 times, for 5 min each time.
(5) The tissue obtained from step (4) was treated with supercritical carbon dioxide at a pressure of 30 MPa, a temperature of 30° C., and a carbon dioxide flow rate of 6 L/min for 30 min.
(6) The tissue obtained from step (5) was treated with a solution of 1 mg/mL EDC and 0.5 mg/mL NHS for 1 h, collected, and washed with deionized water 3 times, for 5 min each time.
(7) The tissue obtained from step (6) was soaked in 100% ethanol for 5 min, then the liquid was changed to deionized water for 5 min, and this process was repeated 5 times, and then soaked in deionized water for 30 min.
(8) The tissue obtained from step (7) underwent lyophilizing, first pre-frozen at −70° C. for 4 h, then placed in a lyophilizer. In the second stage, a vacuum was applied, and the lyophilizing process was same as Example 1.

### Comparative Example 5

This comparative example provides an ordered collagen scaffold material, and the preparation method is as follows (a combination treatment of electrical pulse stimulation and ultrasound stimulation was replaced by the trypsin treatment).
(1) A fresh bovine fascial tissue was taken, and the muscle and adipose tissues were physically removed, and then soaked in deionized water at 4° C. for 2 h, with the water being changed every half hour.
(2) The tissue was soaked in 1% trypsin for 2.5 h, the buffer was 25 mmol/L Tris-HCl (pH 7.6), and washed with deionized water 10 times, for 5 min each time.
(3) The tissue obtained from step (2) was soaked in a 2 M NaOH aqueous solution for 1 h, after which the alkaline solution was removed and the tissue was washed with deionized water 10 times, for 5 min each time.
(4) The tissue obtained from step (3) was then soaked in a mixed solution of ethanol and ethyl acetate (with a volume ratio of 1:2) at 16° C. for 10 h, followed by washing with deionized water 10 times, for 5 min each time.
(5) The tissue obtained from step (4) was treated with supercritical carbon dioxide at a pressure of 30 MPa, a temperature of 30° C., and a carbon dioxide flow rate of 6 L/min for 30 min.
(6) The tissue obtained from step (5) was treated with a solution of 1 mg/mL EDC and 0.5 mg/ml NHS for 1 h, collected, and washed with deionized water 3 times, for 5 min each time.
(7) The tissue obtained from step (6) was soaked in 100% ethanol for 5 min, then the liquid was changed to deionized water for 5 min, and this process was repeated 5 times, and then soaked in deionized water for 30 min.
(8) The tissue obtained from step (7) underwent lyophilizing, first pre-frozen at −70° C. for 4 h, then placed in a lyophilizer. In the second stage, a vacuum was applied, and the lyophilizing process was same as Example 1.

### Comparative Example 6

This comparative example provides an ordered collagen scaffold material, and the preparation method is as follows (a combination treatment of electrical pulse stimulation and ultrasound stimulation was replaced by the papain and DNase I/RNase I treatment).
(1) A fresh bovine fascial tissue was taken, and the muscle and adipose tissues were physically removed, and then soaked in deionized water at 4° C. for 2 h, with the water being changed every half hour.
(2) The tissue was soaked in 300 U/g papain for 0.5 h, the buffer was phosphate buffer with pH=6, and washed with deionized water 5 times, for 10 min each time; and the tissue was treated in 100 U/g DNase I/RNase I mixture for 3 h, then washed with deionized water for 5 times, each time for 10 min.
(3) The tissue obtained from step (2) was soaked in a 2 M NaOH aqueous solution for 1 h, after which the alkaline solution was removed and the tissue was washed with deionized water 10 times, for 5 min each time.
(4) The tissue obtained from step (3) was then soaked in a mixed solution of ethanol and ethyl acetate (with a volume ratio of 1:2) at 16° C. for 10 h, followed by washing with deionized water 10 times, for 5 min each time.
(5) The tissue obtained from step (4) was treated with supercritical carbon dioxide at a pressure of 30 MPa, a temperature of 30° C., and a carbon dioxide flow rate of 6 L/min for 30 min.
(6) The tissue obtained from step (5) was treated with a solution of 1 mg/mL EDC and 0.5 mg/mL NHS for 1 h, collected, and washed with deionized water 3 times, for 5 min each time.
(7) The tissue obtained from step (6) was soaked in 100% ethanol for 5 min, then the liquid was changed to deionized water for 5 min, and this process was repeated 5 times, and then soaked in deionized water for 30 min.
(8) The tissue obtained from step (7) underwent lyophilizing, first pre-frozen at −70° C. for 4 h, then placed in a lyophilizer. In the second stage, a vacuum was applied, and the lyophilizing process was same as Example 1.

### Comparative Example 7

This comparative example provides an ordered collagen scaffold material, and the preparation method is as follows (a combination treatment of electrical pulse stimulation and ultrasound stimulation was replaced by the treatment of sodium dodecyl sulfate and sodium deoxycholate).
(1) A fresh bovine fascial tissue was taken, and the muscle and adipose tissues were physically removed, and then soaked in deionized water at 4° C. for 2 h, with the water being changed every half hour.
(2) The tissue was treated in a 0.2% of sodium dodecyl sulfonate aqueous solution for 0.5 h, and treated with 3% of sodium deoxycholate for 2 h, and washed with deionized water 10 times, for 5 min each time.
(3) The tissue obtained from step (2) was soaked in a 2 M NaOH aqueous solution for 1 h, after which the alkaline solution was removed and the tissue was washed with deionized water 10 times, for 5 min each time.
(4) The tissue obtained from step (3) was then soaked in a mixed solution of ethanol and ethyl acetate (with a volume ratio of 1:2) at 16° C. for 10 h, followed by washing with deionized water 10 times, for 5 min each time.
(5) The tissue obtained from step (4) was treated with supercritical carbon dioxide at a pressure of 30 MPa, a temperature of 30° C., and a carbon dioxide flow rate of 6 L/min for 30 min.
(6) The tissue obtained from step (5) was treated with a solution of 1 mg/mL EDC and 0.5 mg/mL NHS for 1 h, collected, and washed with deionized water 3 times, for 5 min each time.
(7) The tissue obtained from step (6) was soaked in 100% ethanol for 5 min, then the liquid was changed to deionized water for 5 min, and this process was repeated 5 times, and then soaked in deionized water for 30 min.
(8) The tissue obtained from step (7) underwent lyophilizing, first pre-frozen at −70° C. for 4 h, then placed in a lyophilizer. In the second stage, a vacuum was applied, and the lyophilizing process was same as Example 1.

### Comparative Example 8

This comparative example provides an ordered collagen scaffold material, and the preparation method is as follows (with single ultrasound stimulation).
(1) A fresh bovine fascial tissue was taken, and the muscle and adipose tissues were physically removed, and then soaked in deionized water at 4° C. for 2 h, with the water being changed every half hour.
(2) The tissue was soaked in water at 4° C. for 15 min, then soaked in water at 16° C., and ultrasonic stimulation was performed with a power of 200 W for 10 min.
(3) Steps (2) were repeated three times.
(4) The tissue obtained from step (3) was soaked in a 2 M NaOH aqueous solution for 1 h, after which the alkaline solution was removed and the tissue was washed with deionized water 10 times, for 5 min each time.
(5) The tissue obtained from step (4) was then soaked in a mixed solution of ethanol and ethyl acetate (with a volume ratio of 1:2) at 16° C. for 10 h, followed by washing with deionized water 10 times, for 5 min each time.
(6) The tissue obtained from step (5) was treated with supercritical carbon dioxide at a pressure of 30 MPa, a temperature of 30° C., and a carbon dioxide flow rate of 6 L/min for 30 min.
(7) The tissue obtained from step (6) was treated with a solution of 1 mg/mL EDC and 0.5 mg/mL NHS for 1 h, collected, and washed with deionized water 3 times, for 5 min each time.
(8) The tissue obtained from step (7) was soaked in 100% ethanol for 5 min, then the liquid was changed to deionized water for 5 min, and this process was repeated 5 times, and then soaked in deionized water for 30 min.
(9) The tissue obtained from step (8) underwent lyophilizing, first pre-frozen at −70° C. for 4 h, then placed in a lyophilizer. In the second stage, a vacuum was applied, and the lyophilizing process was same as Example 1.

### Comparative Example 9

This comparative example provides an ordered collagen scaffold material, and the preparation method is as follows (with single electrical pulse stimulation).
(1) A fresh bovine fascial tissue was taken, and the muscle and adipose tissues were physically removed, and then soaked in deionized water at 4° C. for 2 h, with the water being changed every half hour.
(2) After soaking the tissue in water at 4° C. for 15 min, the tissue was subjected to electrical pulse stimulation using electrodes. The stimulation voltage was set at 180 V, with a pulse length of 200 ms and a pulse interval of 1 s, for a total of 100 pulses.
(3) Steps (2) were repeated three times.
(4) The tissue obtained from step (3) was soaked in a 2 M NaOH aqueous solution for 1 h, after which the alkaline solution was removed and the tissue was washed with deionized water 10 times, for 5 min each time.
(5) The tissue obtained from step (4) was then soaked in a mixed solution of ethanol and ethyl acetate (with a volume ratio of 1:2) at 16° C. for 10 h, followed by washing with deionized water 10 times, for 5 min each time.
(6) The tissue obtained from step (5) was treated with supercritical carbon dioxide at a pressure of 30 MPa, a temperature of 30° C., and a carbon dioxide flow rate of 6 L/min for 30 min.
(7) The tissue obtained from step (6) was treated with a solution of 1 mg/mL EDC and 0.5 mg/mL NHS for 1 h, collected, and washed with deionized water 3 times, for 5 min each time.
(8) The tissue obtained from step (7) was soaked in 100% ethanol for 5 min, then the liquid was changed to deionized water for 5 min, and this process was repeated 5 times, and then soaked in deionized water for 30 min.
(9) The tissue obtained from step (8) underwent lyophilizing, first pre-frozen at −70° C. for 4 h, then placed in a lyophilizer. In the second stage, a vacuum was applied, and the lyophilizing process was same as Example 1.

### Test Example 1

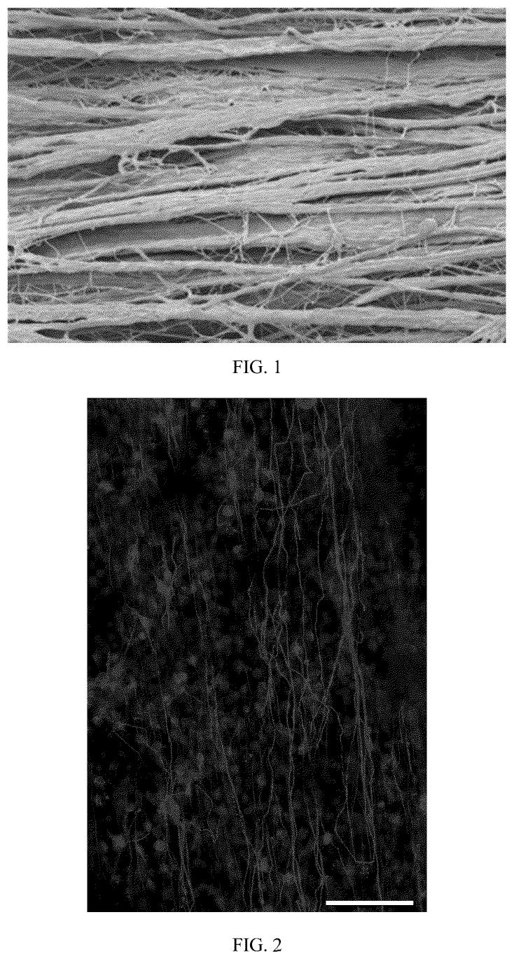

The product prepared in Example 1 was subjected to scanning electron microscopy analysis, as shown in FIG. 1. As can be seen from the figure, the collagen scaffold material involved in the present application has an ordered parallel arrangement of fiber structure.

### Test Example 2

To explore whether neurons can be grown on the collagen scaffold material involved in the present application, taking the product prepared in Example 1 as an example, and the specific operations are as follows. The spinal cord neural stem cells were isolated from embryonic rats at 14 days of gestation and cultured as neurospheres in DMEM/F12 medium supplemented with 20 ng/ml basic fibroblast growth factor (bFGF) and 20 ng/mL epidermal growth factor (EGF) for 7 days. Subsequently, 1000 cells were seeded onto a scaffold material with a length of 1 cm and cultured for 7 days in cell culture medium containing 10 ng/ml neurotrophin-3 (NT3) and 20 ng/ml brain-derived neurotrophic factor (BDNF). The cells were then subjected to immunofluorescence staining using the Tuj1 antibody, and the adhesion and growth of Tuj1-positive neuron cells on the scaffold material were analyzed. The results are showed in FIG. 2 (scale bar: 100 μm). As can be seen from the figure, the rat neuron cells are capable of growing in an ordered manner along the extension direction of the collagen scaffold material in the present application.

### Test Example 3

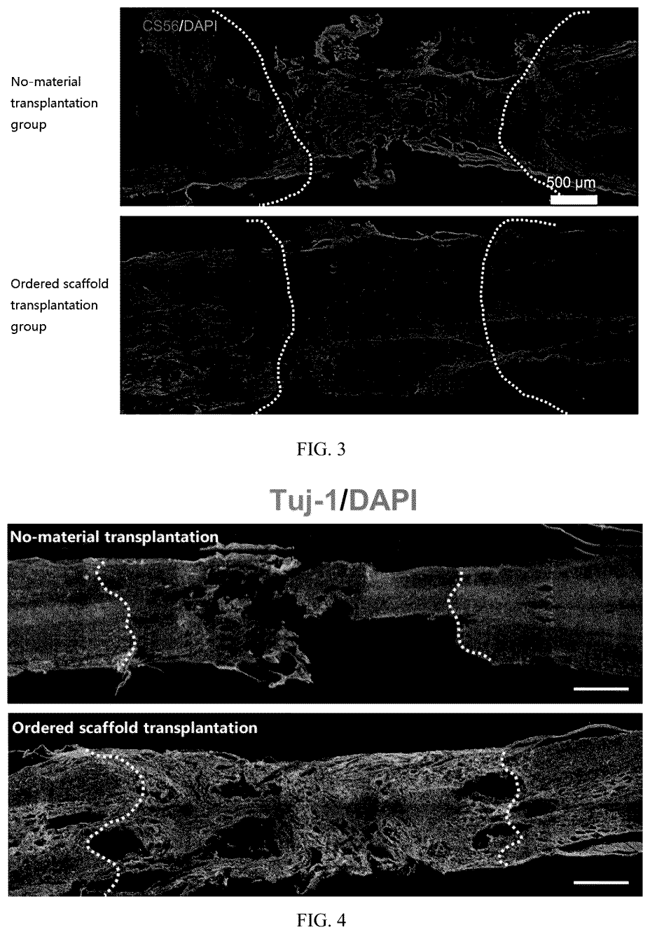

The products prepared in Examples 1-6 and Comparative Examples 1-9 were tested for the following properties, respectively. The specific test methods or criteria are shown below, and the results are shown in Table 1.
(1) Nucleic acid content: the test was performed in accordance with the method specified in YY/T 0606.25-2014.
(2) Fat content: the test was performed in accordance with the method specified in the First Method of GB/T 5009.6-2010.
(3) Standard collagen content: the test was performed in accordance with determination method of hydroxyproline in Appendix B of YY/T 1511-2017.
(4) Cytotoxicity: the test was performed in accordance with the method specified in GB/T 16886.5-2017.
(5) Detection of scar proportion after transplantation in the spinal cord injury area:
(5.1) Construction of a 5 mm complete transection spinal cord injury model in the thoracic segment in adult rats (SD Rats, 10 weeks old): the rats were anaesthetized using sodium pentobarbital, the dorsal spine at the thoracic segment T8 was opened, a 5 mm defect was created in the spinal cord tissue, hemostasis was performed on the tissue, and different materials were transplanted.
(5.2) The ordered collagen scaffold materials prepared from each group were transplanted into the spinal cord injury site, with injured rats without material transplantation serving as controls, and there were 9 rats in each group. After 72 days of transplantation, the animals were euthanized, and the spinal cord tissues were extracted and soaked in a 4% formaldehyde solution for fixation for 48 h. Subsequently, a 30% sucrose solution was added for dehydration for 72 h. The tissues were then embedded and frozen for sectioning, with a section thickness of 20 μm. The sections were subjected to immunofluorescence staining with the Anti-Chondroitin Sulfate antibody (CS-56), produced by Abcam number: ab (product 11570). Simultaneously, DAPI (4′,6-diamidino-2-phenylindole) was used to stain the nuclei, and the proportion of CS-56 positive area in the spinal cord region was calculated as the scar proportion after transplantation in the injury area. The results from the no-material transplantation group and the Example 1 material transplantation group are shown in FIG. 3 (CS-56 represents chondroitin sulfate, indicating the formation of glial scars, and DAPI indicates nuclear staining. The dashed line indicates the edge of the injury).

As can be seen from the results of the data in FIG. 3 and Table 1, the ordered collagen scaffold material involved in the present application significantly reduces the deposition of glial scar after transplanted to the spinal cord injury site, which is critical for repairing spinal cord injury.
(7) Detection of nerve regeneration length after transplantation in spinal cord injury area.

The sections were subjected to immunofluorescence staining using the Anti-beta III Tubulin antibody (Tuj1), produced by Abcam (product number ab18207). Simultaneously, DAPI was used to stain the nuclei, and the length of Tuj1-positive nerve axons in the spinal cord region within the injury area was quantified as the nerve regeneration length after transplantation. The results from the no-material transplantation group and the Example 1 material transplantation group are shown in FIG. 4 (Tuj-1 represents nerve axons, DAPI indicates nuclear staining, and the scale bar is 1 mm).

As can be seen from the results of the data in FIG. 4 and Table 1, the ordered collagen scaffold material involved in the present application can effectively promote the regeneration of nerve axons at the injury site after transplanted to the spinal cord injury site.

TABLE 1

| | | | | | Average scar | |
|---|---|---|---|---|---|---|
| | | | | | tissue | Average nerve |
| | | | | | proportion | regeneration |
| | Average | | | | after | length after |
| | nucleic | Average | Average | | transplantation | transplantation |
| | acid | fat | collagen | | in the spinal | in spinal cord |
| | content | content | content | Cytotoxicity | cord injury | injury area |
| Group | (ng/mg) | (%) | (%) | (grade) | area (%) | (μm) |
| Example 1 | 5.23 | 0.12 | 99.74 | 0 | 3.28 | 2924.50 |
| Example 2 | 6.18 | 0.14 | 99.58 | 0 | 4.12 | 2721.56 |
| Example 3 | 7.14 | 0.19 | 98.50 | 0 | 5.28 | 2636.06 |
| Example 4 | 6.45 | 0.41 | 97.36 | 0 | 6.36 | 1841.83 |
| Example 5 | 7.16 | 0.49 | 96.30 | 0 | 6.92 | 1731.78 |
| Example 6 | 5.89 | 0.14 | 99.34 | 0 | 14.35 | 1264.87 |
| Comparative | 6.31 | 0.58 | 96.12 | 0 | 7.41 | 1539.30 |
| Example 1 | | | | | | |
| Comparative | 7.35 | 0.51 | 94.07 | 0 | 8.72 | 1423.19 |
| Example 2 | | | | | | |
| Comparative | 5.82 | 0.16 | 96.15 | 0 | 10.32 | 1176.61 |
| Example 3 | | | | | | |
| Comparative | 10.69 | 0.46 | 94.17 | 1 | 10.62 | 923.28 |
| Example 4 | | | | | | |
| Comparative | 9.85 | 0.73 | 93.14 | 1 | 12.12 | 834.25 |
| Example 5 | | | | | | |
| Comparative | 12.28 | 0.86 | 92.12 | 1 | 15.82 | 751.99 |
| Example 6 | | | | | | |
| Comparative | 22.28 | 0.98 | 90.85 | 0 | 18.26 | 623.08 |
| Example 7 | | | | | | |
| Comparative | 9.21 | 0.82 | 93.22 | 0 | 10.23 | 1222.49 |
| Example 8 | | | | | | |
| Comparative | 15.76 | 0.92 | 91.17 | 0 | 14.72 | 827.81 |
| Example 9 | | | | | | |

(8) Animal motor behavior score (BBB Score)

The motor behavior of rats in each group was scored (BBB score, see Table 2 for evaluation criteria) after the transplantation of the ordered collagen scaffold material. A higher score indicates better recovery of motor function, and the results are shown in Table 3:

| Score | Assessment criteria |
|---|---|
| 0 | No hindlimb movement. |
| 1 | Mild movement in one or two hindlimb joints (with a motion range of <50%), |
| | typically hip and knee joints. |
| 2 | Significant movement in one joint (with a motion range of>50%), or significant |
| | movement in one joint combined with mild movement in another joint. |
| 3 | Significant movement in two joints, typically hip and knee joints. |
| 4 | Mild movement in three hindlimb joints (hip, knee, and ankle). |
| 5 | Significant movement in one of three joints (typically hip) combined with mild |
| | movement in the other two (knee and ankle). |
| 6 | Significant movement in two of three joints combined with mild movement in the |
| | third. |
| 7 | Significant movement in three joints. |
| 8 | Plantar placement of the paw without weight-bearing. |
| 9 | Stable plantar placement with weight-bearing posture, or |
| | occasional/frequent/persistent dorsiflexion walking without plantar weight-bearing. |
| 10 | Occasional weight-bearing hindlimbs standing or walking (plantar touches the |
| | ground with loading weight, plantar touches the ground again when hindlimbs |
| | move forward) and no coordination between forelimbs and hindlimbs. |
| 11 | Frequent to persistent standing or walking without coordination between forelimbs |
| | and hindlimbs. |
| 12 | Frequent to persistent standing or walking with occasional coordination between |
| | forelimbs and hindlimbs. |
| 13 | Frequent to persistent weight-bearing walking with coordination between forelimbs |
| | and hindlimbs. |
| 14 | Persistent weight-bearing walking, coordinated movement of forelimbs and hind |
| | limbs, rotation on paw touching the ground and lifting (hind paws rotate externally |
| | or internally on touching the ground or lifting), frequent paw-standing on the |
| | ground while rotating, occasional dorsal standing. |
| 15 | Persistent weight-bearing walking, coordinated movement of forelimbs and hind |
| | limbs, occasional toe extension of the hindlimbs during forward movement (no toe |
| | rubbing sound when toes move forward), toes parallel to body direction when |
| | moving forward. |
| 16 | Persistent weight-bearing walking, coordinated movement of forelimbs and hind |
| | limbs, frequent toe extension of the hindlimbs during forward movement (more |
| | than half without foot-dragging sounds), toes parallel to body direction when |
| | moving forward, and rotate when lifting. |
| 17 | Persistent, coordinated plantar walking with frequent toe extension of the hindlimbs |
| | during forward movement, and the hindlimbs move in a direction parallel but |
| | opposite to the body when lifting and forward movement. |
| 18 | Persistent, coordinated plantar walking with continuous toe extension of the |
| | hindlimbs during forward movement, and the hindlimbs move in a direction parallel |
| | but opposite to the body when lifting and forward movement. |
| 19 | Persistent, coordinated plantar walking with continuous toe extension of the |
| | hindlimbs during forward movement, and the hindlimbs move in a direction parallel |
| | but opposite to the body when lifting and forward movement, and the tail drops. |
| 20 | Persistent, coordinated plantar walking with continuous toe extension of the |
| | hindlimbs during forward movement, and the hindlimbs move in a direction parallel |
| | but opposite to the body when lifting and forward movement, the tail drops, and the |
| | body is unstable (the center of the body weight is shifted sideways during the |
| | movement and there are the phenomena of tilting, swaying and slipping). |
| 21 | Persistent, coordinated plantar walking with continuous toe extension of the |
| | hindlimbs during forward movement, and the hindlimbs move in a direction parallel |
| | but opposite to the body when lifting and forward movement, the tail drops, and the |
| | body is constant stability with a coordinated pelvic and tail movement. |

TABLE 3

| | 7 | 14 | 21 | 28 | 35 | 42 | 49 | 56 | 63 | 72 |
|---|---|---|---|---|---|---|---|---|---|---|
| Group | days | days | days | days | days | days | days | days | days | days |
| Control | 0.2 | 0.6 | 0.9 | 1.3 | 1.4 | 1.7 | 1.9 | 2.1 | 2.3 | 2.3 |
| group | | | | | | | | | | |
| Example 1 | 0.4 | 1.3 | 1.9 | 2.6 | 3.6 | 4.1 | 4.7 | 5.0 | 5.2 | 5.2 |
| Example 2 | 0.2 | 1.2 | 1.8 | 2.3 | 3.2 | 4.0 | 4.4 | 4.7 | 5.0 | 5.0 |
| Example 3 | 0.1 | 1.1 | 1.8 | 2.1 | 3.1 | 3.9 | 4.3 | 4.6 | 4.8 | 4.9 |
| Example 4 | 0.2 | 1.1 | 1.7 | 2.0 | 3.0 | 3.8 | 4.2 | 4.4 | 4.7 | 4.8 |
| Example 5 | 0.3 | 1.0 | 1.6 | 1.9 | 2.8 | 3.7 | 4.1 | 4.3 | 4.6 | 4.7 |
| Example 6 | 0.3 | 1.0 | 1.6 | 1.9 | 2.6 | 3.6 | 3.9 | 4.7 | 4.8 | 4.9 |
| Comparative | 0.4 | 0.9 | 1.4 | 1.9 | 2.7 | 3.6 | 4.0 | 4.4 | 4.7 | 4.8 |
| Example 1 | | | | | | | | | | |
| Comparative | 0.3 | 0.8 | 1.3 | 2.0 | 2.6 | 3.7 | 4.1 | 4.6 | 4.8 | 4.8 |
| Example 2 | | | | | | | | | | |
| Comparative | 0.4 | 0.9 | 1.4 | 2.0 | 2.7 | 3.7 | 4.0 | 4.7 | 4.9 | 5.0 |
| Example 3 | | | | | | | | | | |
| Comparative | 0.3 | 0.7 | 1.0 | 1.2 | 1.6 | 1.8 | 2.0 | 2.2 | 2.6 | 2.8 |
| Example 4 | | | | | | | | | | |
| Comparative | 0.4 | 0.6 | 1.1 | 1.3 | 1.4 | 1.9 | 2.1 | 2.3 | 2.7 | 2.9 |
| Example 5 | | | | | | | | | | |
| Comparative | 0.6 | 0.7 | 1.2 | 1.4 | 1.6 | 2.0 | 2.2 | 2.4 | 2.8 | 3.0 |
| Example 6 | | | | | | | | | | |
| Comparative | 0.7 | 0.8 | 1.3 | 1.6 | 1.7 | 2.2 | 2.3 | 2.6 | 2.9 | 3.1 |
| Example 7 | | | | | | | | | | |
| Comparative | 0.6 | 0.7 | 1.2 | 1.4 | 1.8 | 2.1 | 2.4 | 2.4 | 2.8 | 3.0 |
| Example 8 | | | | | | | | | | |
| Comparative | 0.4 | 0.8 | 1.1 | 1.4 | 1.7 | 2.0 | 2.3 | 2.6 | 3.0 | 3.2 |
| Example 9 | | | | | | | | | | |

As can be seen from the results of the data in Table 3, compared with Comparative Examples 1-6, the ordered collagen scaffold material prepared by the preparation method involved in the present application can significantly improve the recovery of motor function after transplanted to the spinal cord injury site.

### Application Example 1

This application example provides an ordered collagen scaffold material loaded with the drug Cetuximab, and the preparation method is as follows.

The ordered nerve regeneration collagen scaffold material (with a length of 5 mm and a diameter of 2 mm) prepared in Example 1 was added with 10 μL of Cetuximab (purchased from Merck KGaA, with a commercial specification of 100 mg/20 mL/bottle) with a concentration of 2 μg/μL, so that all was absorbed by the nerve regeneration collagen scaffold, and incubated at 4° C. for 60 min to obtain the ordered collagen scaffold material with the loading of the drug Cetuximab.

### Test Example 4

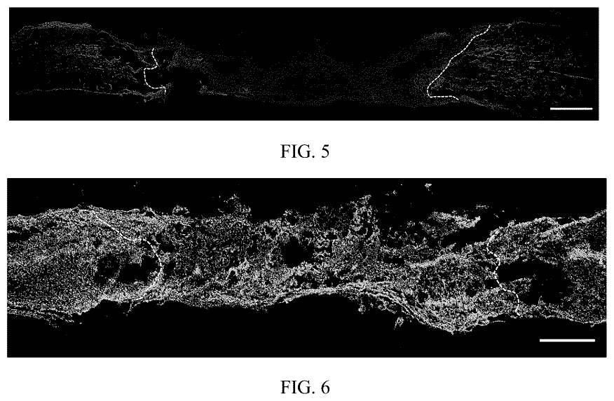

The products prepared from the above application examples were tested for the following properties, and the specific test methods or criteria are shown below, and the results are shown in Table 4.
(1) Detection of scar proportion after transplantation in the spinal cord injury area: the ordered collagen scaffold material loaded with the drug Cetuximab prepared in the Application Example was transplanted to the spinal cord injury site in the same manner as in Test Example 3. The results of the Application Example material after the transplantation and repairing are shown in FIG. 5 (CS-56 represents chondroitin sulphate, indicating the formation of gelatinous scarring, and DAPI represents cell nuclear staining. The dashed line indicates the edge of the injury).
(2) Detection of nerve regeneration length after transplantation in spinal cord injury area: the ordered collagen scaffold material loaded with the drug Cetuximab prepared in the Application Example was transplanted to the spinal cord injury site in the same manner as in Test Example 3. The results of the Application Example material after the transplantation and repairing are shown in FIG. 6 (Tuj-1 represents nerve axons, DAPI represents cell nuclei, scale bar: 1 mm).

As can be seen from the results of the data in Table 4, the ordered collagen scaffold material involved in the present application can further promote the regeneration of nerve axons at the injury site after transplanted to the spinal cord injury site.

TABLE 4

| | | Average nerve regeneration |
|---|---|---|
| | Average scar proportion | length after |
| | after transplantation | transplantation |
| | in the spinal cord | in spinal cord |
| Group | injury area (%) | injury area (μm) |
| Application | 2.76 | 3162.42 |
| Example | | |

The applicant declares that technical solutions in the present application are illustrated by the above examples in the present application, but the present application is not limited to the above examples, that is, the present application does not necessarily rely on the above examples to be implemented. Those skilled in the art should understand that any improvement of the present application, the equivalent substitution of raw materials, the addition of auxiliary ingredients, and the selection of specific methods of the products in the present application shall fall within the protection scope and disclosure scope of the present application.

The above describes the preferred embodiments of the present application in detail. However, the present application is not limited to the specific details in the above embodiments, and within the scope of the technical conception of the present application, a plurality of simple variations of the technical solution of the present application may be performed, and all simple variations fall within the protection scope of the present application.

It should also be noted that each of specific technical features described in the above specific embodiments can be combined in any suitable way without contradiction, and in order to avoid unnecessary repetition, the present application will not separately describe the various possible combinations.

While the disclosure has been described with reference to a preferred embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for preparing an ordered collagen scaffold material, the method comprising the following steps:
(1) subjecting a mammalian fascial tissue to a combined treatment of electrical pulse stimulation and ultrasound stimulation;
(2) soaking a product after treatment in an alkaline solution;
(3) soaking a product after alkali solution treatment in an organic solution for a preliminary degreasing treatment, and performing a secondary degreasing treatment with supercritical carbon dioxide;
(4) cross-linking a product after degreasing treatment with a chemical cross-linking agent;
(5) fixing and molding a product after cross-linking treatment; and
(6) lyophilizing a product after fixing and molding to obtain the ordered collagen scaffold material.

2. The method according to claim 1, wherein in step (1), the combined treatment is performed with electrical pulse stimulation and ultrasound stimulation for 1-5 times alternately or sequentially;
wherein the electrical pulse stimulation is to treat a fascia tissue with an electrode, a stimulation voltage is set to 10-100 V, a pulse length is set to 10-250 ms, and a pulse interval is set to 1-7 s, and 10-100 pulses are stimulated stimulation each time; and
wherein the ultrasound stimulation is performed with a power of 50-200 W for a period of 1-10 min.

3. The method according to claim 1, wherein in step (2), the alkali solution is an aqueous NaOH solution of 0.5-3 M, and the soaking is performed for a period of 0.5-3 h, and the soaking is performed at a temperature of 2-10° C.; and
wherein a product after soaking is further washed.

4. The method according to claim 1, wherein in step (3), the organic solution comprises a combination of ethanol and ethyl acetate, and a volume ratio of ethanol to ethyl acetate is 1:1-1:4; in step (3), the soaking is performed for a period of 15-30 h, and the soaking is performed at a temperature of 20-30° C.; and a product after soaking is cleaned further.

5. The method according to claim 1, wherein in the secondary degreasing treatment with supercritical carbon dioxide of step (3), the supercritical carbon dioxide has a pressure of 5-40 MPa at a temperature of 25-55° C.; and wherein carbon dioxide has a flow rate of 0.5-12 L/min, and the secondary degreasing treatment is performed for a period of 20-40 min.

6. The method according to claim 1, wherein in step (4), the chemical cross-linking agent comprises N-hydroxysuccinimide and 1-(3-dimethylaminopropyl)-3-ethyl carbodiimide; and the cross-linking treatment is performed at a temperature of 30-40° C. for a period of 0.5-1.5 h; a product after cross-linking is further washed; and
wherein in step (5), the fixing and molding is performed in a way that the product is alternately soaking in ethanol solution and water for a total of 3-8 times, and each soaking is performed for a period of 3-8 min; wherein an ethanol concentration of the ethanol solution is 75-100%.

7. The method according to claim 1, wherein in step (6), the lyophilizing is performed firstly by pre-freezing, and a procedure of lyophilizing is as follows: a first stage: −35 to −25° C. for 1-5 h; a second stage: −25 to −15° C. for 0.5-1.5 h; a third stage: −25 to −15° C. for 5-15 h; a fourth stage: −15 to −5° C. for 0.5-1.5 h; the fifth stage: −15 to −5° C. for 10-20 h; a sixth stage: −5 to −5° C. for 1-5 h; a seventh stage: 5 to 15° C. for 1-5 h; an eighth stage: 10 to 20° C. for 1-5 h; a ninth stage: 20 to 30° C. for 1-5 h; and vacuuming is performed in the second stage.

8. An ordered collagen scaffold material prepared by the method of claim 1.

9. A method for repairing spinal cord injury, comprising using the ordered collagen scaffold material of claim 8.

10. A product for repairing spinal cord injury, comprising:
the ordered collagen scaffold material of claim 8 and Cetuximab loaded on the ordered collagen scaffold material;
wherein the product for repairing spinal cord injury is prepared by a preparation method comprising the following steps:
soaking the ordered collagen scaffold material in Cetuximab solution and incubating at 0-30° C. for 20-60 min.
